SEQUENCE LISTING (9 sequences)

SEQ ID NO: 1 - DNA, 29 bp, Artificial Sequence
  Synthetic
  misc_feature (29)..(29): 3′-FAM fluorophore
tagccctaaagctatttcggagagaacca

SEQ ID NO: 2 - DNA, 21 bp, Artificial Sequence
  Synthetic
gagaaattccaaacgaacttg

SEQ ID NO: 3 - DNA, 21 bp, Artificial Sequence
  Synthetic
cagtgctctacctccatcatt

SEQ ID NO: 4 - DNA, 29 bp, Artificial Sequence
  Synthetic
  misc_feature (1)..(1): 5′-6-FAM fluorophore
  misc_feature (29)..(29): 3′-TAMRA fluorophore
tggttctctccgaaatagctttagggcta

SEQ ID NO: 5 - DNA, 194 bp, Artificial Sequence
  Synthetic
tcatgcaagtcgagcgatggagaaattccaaacgaacttgggggttctgagaggaaggtggtagagcactgtttcggcatctgaggagcacgagacggcaggctcgagaatgatggaggtagagcactgaaaaggaagattaataccgcatagagaatgttatcacgggagacaagtagcgtgaaggatgacgg

SEQ ID NO: 6 - DNA, 27 bp, Artificial Sequence
  Synthetic
acgaattcgtgccagcagccgcggtaa

SEQ ID NO: 7 - DNA, 27 bp, Artificial Sequence
  Synthetic
  misc_feature (7)..(7): Inosine Residue
tggaatncggttaccttgttacgactt

SEQ ID NO: 8 - DNA, 27 bp, Artificial Sequence
  Synthetic
gctcacccagaaacgctggtgaaagta

SEQ ID NO: 9 - DNA, 27 bp, Artificial Sequence
  Synthetic
cgcaacgttgttgccattgctacaggc

(19) United States
(12) Patent Application Publication
(10) Pub. No.: US 2022/0325270 A1
(43) Pub. Date: Oct. 13, 2022

(54) AN ON-CHIP BIFURCATED CONTINUOUS FIELD-FLOW FRACTIONATION TECHNOLOGY FOR NUCLEIC ACID ISOLATION

(71) Applicant: University of Notre Dame du Lac, Notre Dame, IN (US)

(72) Inventors: Chenguang Zhang, South Bend, IN (US); Gongchen Sun, South Bend, IN (US); Satyajyoti Senapati, South Bend, IN (US); Hsueh-Chia Chang, South Bend, IN (US)

(21) Appl. No.: 17/639,198

(22) PCT Filed: Oct. 2, 2020
(86) PCT No.: PCT/US2020/053926

§ 371 Date: Feb. 28, 2022

Related U.S. Application Data
(60) Provisional application No. 62/915,402, filed on Oct. 15, 2019.
(60) Provisional application No. 62/905,567, filed on Sep. 25, 2019.

(51) Int. Cl. C12N 15/10

(52) U.S. Cl. CPC C12N 15/1003

(57) ABSTRACT

Described herein is a bifurcated continuous field-flow fractionation (BCFFF) chip for high-yield and high-throughput nucleic acid extraction and purification. BCFFF uses a membrane ionic transistor to sustain low-ionic strength in a localized region at a junction, such that the resulting high field can selectively isolate high-charge density nucleic acids from the main flow channel and insert them into a standardized buffer in a side channel that bifurcates from the junction. The BCFFF platform can be used for isolation of both long dsDNAs and short miRNAs, without changing the device configuration or the operation protocol. BCFFF results in high-efficiency (>85%) concentration-independent DNA extraction and 40% net qRT-PCR miRNA yield from plasma, which is significantly higher than any other commercial liquid and solid extraction technologies.

### CROSS REFERENCE TO RELATED APPLICATIONS

This application is a U.S. national phase of International Application No. PCT/US2020/053926, filed Oct. 2, 2020, which claims priority to U.S. Provisional Patent Application Nos. 62/905,567, filed on Sep. 25, 2019, and 62/915,402, filed on Oct. 15, 2019, the entire contents of each which are incorporated by reference herein.

### FEDERALLY SPONSORED RESEARCH

This invention was made with U.S. government support under grant CA206904 awarded by the National Institutes of Health. The U.S. government has certain rights in the invention

### REFERENCE TO SEQUENCE LISTING

This application is filed with a Computer Readable Form of a Sequence Listing in accordance with 37 C.F.R. § 1.821(c). The text file submitted by EFS, “092012-9133-US04_sequence_listing_28 Feb. 2022_ST25.txt,” was created on Feb. 28, 2022, has a file size of 2.60 Kbytes, and is incorporated by reference herein in its entirety.

### TECHNICAL FIELD

Described herein is a bifurcated continuous field-flow fractionation (BCFFF) chip for high-yield and high-throughput nucleic acid extraction and purification. BCFFF uses a membrane ionic transistor to sustain low-ionic strength in a localized region at a junction, such that the resulting high field can selectively isolate high-charge density nucleic acids from the main flow channel and insert them into a standardized buffer in a side channel that bifurcates from the junction. The BCFFF platform can be used for isolation of both long dsDNAs and short miRNAs, without changing the device configuration or the operation protocol. BCFFF results in high-efficiency (>85%) concentration-independent DNA extraction and 40% net qRT-PCR miRNA yield from plasma, which is significantly higher than any other commercial liquid and solid extraction technologies.

### BACKGROUND

The detection and quantification of nucleic acids play essential roles in various applications, such as clinical diagnostics, food safety, forensic analysis, and environmental monitoring. In the last two decades, the discovery of host nucleic acid biomarkers (mutations in coding DNA/mRNA and irregular expressions of noncoding miRNA) for different diseases has opened up numerous new and future applications. In such biomarker applications, particularly with non-coding miRNAs, accurate nucleic acid quantification in bodily fluids becomes the overriding requisite. The current nucleic acid analysis technologies, like PCR, microarray, and sequencing can provide sensitive and selective quantification after the nucleic acid molecules are extracted from physiological samples (mostly blood) rich in various PCR and hybridization inhibitors and inserted into a standardized reagent buffer. The standardized buffer provides a baseline for normalization for the various sequence-, ionic strength- and pH-dependent quantitative nucleic acid reaction assays. Removal of inhibitory agents like metal cations, proteins, nucleases, proteinase, etc. in the physiological samples would also remove the quantitative bias they introduce. Consequently, a high-yield pre-treatment step to extract and purify the nucleic acid targets and insert them into a standardized buffer becomes the key step for sensitive and accurate quantification, independent of the actual detection platform. With these high-yield pre-treatment steps, welcomed features like absolute quantification and cross-platform comparison become possible.

Current extraction methods are based on liquid extraction into immiscible liquids and solid extraction by high-affinity binding columns. Immiscible organic solvents like phenol and chloroform are used during such extraction steps and they must be removed before further analysis. These multiple extraction, binding, and washing steps render the entire procedure labor-intensive, low-throughput and, most importantly, low-yield. Efforts have been made to transfer such binding/washing extraction principles onto microfluidic lab-on-a-chip systems. Recent advances in integration and novel extraction methods show improvement of extraction efficiency, especially for large nucleic acid molecules. However, the low binding efficiency and solubility of short nucleic acids, like 20-base miRNAs, often stipulate much larger sorbent volume and extracting liquid than can be accommodated by a microfluidic chip. High-affinity absorbing materials have been developed with optimized elution buffer but they remain inadequate, with extraction efficiencies below 20%. Moreover, the extraction efficiency for each kit is concentration-dependent, with the yield going down at lower concentrations. This greatly complicates the quantification effort, as normalization with respect to a housekeeping molecule becomes inaccurate and extensive calibration is necessary. Multiple and non-continuous operation steps of the current microfluidic-based extraction modules render their integration with downstream PCR or other detection modules extremely difficult. Clearly, a high-throughput and high-yield extraction chip module that can be integrated with downstream steps in an integrated continuous-flow platform would significantly elevate the detection sensitivity, quantification accuracy, and usability of nucleic acid analysis technologies.

We report such an on-chip microfluidic extraction technology here that can isolate nucleic acids from an inhibitor-rich plasma sample and insert them into a standardized PCR buffer at high yield and throughput. We develop a field-flow fractionation design, which extracts charged nucleic acids from a continuous flow by electrophoresis. The isolation performance of our method is significantly enhanced for high-mobility nucleic acids through the creation of a low ionic strength region with a high electric field at the bifurcation junction. The field (˜100 V/cm) is about 10 times higher than in capillary or gel electrophoresis but is still 100 times lower than the value necessary to damage the nucleic acids. The high field allows us to selectively remove nucleic acids from a flowing sample, insert them in the standardized buffer, and yet reject high-mobility cations that are also PCR inhibitors. The low ionic strength region on the chip is created by the ion depletion action of a gated membrane ionic transistor, reported in our earlier publications, which allows easy control of the range and intensity of the ion-depleted zone. Unlike conventional electrokinetic modules based on external ion concentration polarization of a passive ion-selective membrane without through flow, our design combines the versatility of a gated membrane ionic transistor and the hydrodynamic drag to achieve the continuous field-flow fractionation design. The hydrodynamic drag removes larger molecules with weaker charge density (e.g. the proteins) in the throughflow, while the high electric field extracts the target nucleic acids from protein inhibitors in the main flow channel and inserts them into a bifurcated channel without the cation inhibitors. Such selective extraction from both high-mobility and low-mobility contaminants is difficult to achieve in standard single-channel field-flow fractionation design. Moreover, we do not rely on the transverse gradient of the flow field and hence can achieve much higher yield with simpler tuning efforts. The standardized buffer is introduced into the bifurcated channel to complete the continuous extraction and purification process. Our bifurcated continuous field-flow fractionation (BCFFF) design hence exploits not just the high free-space electrophoretic mobility of the nucleic acids but also the different mobility direction of the counterions that bind to them and induce association or dissociation nucleic acid reactions that inhibit PCR. We demonstrate with an integrated chip an extraction yield from plasma higher than 80% for different nucleic acids with different lengths, ranging from long dsDNA fragments to short miRNAs.

### SUMMARY

One embodiment described herein is an apparatus for isolating nucleic acids from a fluid sample comprising the nucleic acids, the apparatus comprising: an inlet, a first outlet, a second outlet and a microfluidic channel fluidly connecting the inlet, the first outlet and the second outlet, wherein the microfluidic channel includes a first channel portion extending from the inlet to a junction, a second channel portion extending from the junction to the first outlet, and a third channel portion extending from the junction to the second outlet; a cation exchange membrane (CEM) positioned across the second channel portion proximate to the junction; a plurality of electrodes adapted to generate an electric field at the junction. In one aspect, the plurality of electrodes include a first terminal positioned within the third channel portion, a second terminal positioned within the second channel portion and a third terminal positioned on the CEM. In another aspect, the microfluidic channel has a thickness between about 1 μm and about 100 μm.

Another embodiment described herein is a method for isolating nucleic acids from a fluid sample comprising the nucleic acids, the method comprising: providing the apparatus described herein; filling the microfluidic channel with a buffered solution comprising anions and cations; applying an electric field at the junction of about 0.1 to 10 V/mm thereby causing cations at the junction to migrate away from the junction through the CEM, and causing anions at the junction to migrate away from the junction towards the second outlet, and generating an ion depletion region at the junction; isolating nucleic acids from the fluid sample by: continuously loading the fluid sample into the microfluidic channel through the inlet and removing fluid from the first outlet such that fluids flow from the inlet to the first outlet at a flow rate that permits nucleic acids to electrophoretically migrate from the junction towards the second outlet due to the applied electric field, and to concentrate at the second outlet; and thereafter, collecting concentrated nucleic acids from the second outlet. In one aspect, the ion depletion region has an ionic strength less than about 1.0 mM. In another aspect, the dimension of the ion depletion region is inversely proportional to the applied electric field. In another aspect, the length of the ion depletion region is between about 0.1 mm and about 10 mm. In another aspect, the applied electric field and the flow rate are selected so as to permit at least 80% of the nucleic acids in the fluid sample to electrophoretically migrate from the junction towards the second outlet and concentrate at the second outlet. In another aspect, the flow rate is between about 0.1 μL/min and about 100 μL/min. In another aspect, the nucleic acids comprise one or more of ssDNA, dsDNA, rDNA, cDNA, ssRNA, dsRNA, rRNA, mRNA, tRNA, siRNA, or miRNA. In another aspect, the fluid sample further comprises one or more cationic molecules, and wherein the applied electric field and the flow rate cause the cationic molecules to electropheretically and hydrodynamically migrate from the junction towards the CEM. In another aspect, the cationic molecules comprise ions or proteins. In another aspect, the fluid sample further comprises anionic biomolecules other than nucleic acids, and wherein the flow rate causes the anionic biomolecules to hydrodynamically migrate from the junction towards the CEM.

### BRIEF DESCRIPTION OF THE DRAWINGS

FIG. 1A. The layout of the bifurcated continuous field-flow fractionation (BCFFF) chip.

FIG. 1B. The fabrication method of the chip.

FIG. 1C. Actual picture of the device.

FIG. 1D. Working principle of the continuous field-flow fractionation chip.

FIG. 2. Real-time fluorescence images show the isolation of the fluorescent-labelled ssDNAs. 1: t=134 s; 2: t=269 s; 3: t=435 s; 4: t=571 s; 5: t=766 s; 6: t=947 s; 7: t=1114 s; 8: t=1280 s. The sample loading stops after 1000 s V_{d}=80 V and V_{g}=1V.

FIG. 3A. The extraction efficiency of the fluorescence-labeled ssDNA with different sample loading flow rates (V_{d}: 80 V/V_{g}: 1 V).

FIG. 3B. The extraction efficiency of the fluorescence-labeled ssDNA with different applied voltages (flow rate: 1 μL/min).

FIG. 3C. Simulation of the electric potential and the electric field near the depletion front at different applied voltages.

FIG. 4A. Efficient removal of PCR inhibitors: Calcium ions.

FIG. 4B. Efficient removal of PCR inhibitors: Proteins (GFP).

FIG. 5. Agarose gel electrophoresis results of PCR for (1) rDNA in nucleic acid extracted on chip, (2) Ampicillin-resistance gene in nucleic acid extracted on chip, (3) rDNA in untreated sample, (4) Ampicillin-resistance gene in untreated sample.

FIG. 6A. Evaluation of PCR efficiency by a series of dilution of the sample, the eluted sample has similar PCR efficiency to the inhibitor-free control.

FIG. 6B. C_{q} value from qPCR of the eluted sample with different initial spiked concentrations.

FIG. 7. Yield of cel-mir-39 spiked in human plasma from chip and Qiagen MiReasy Kit.

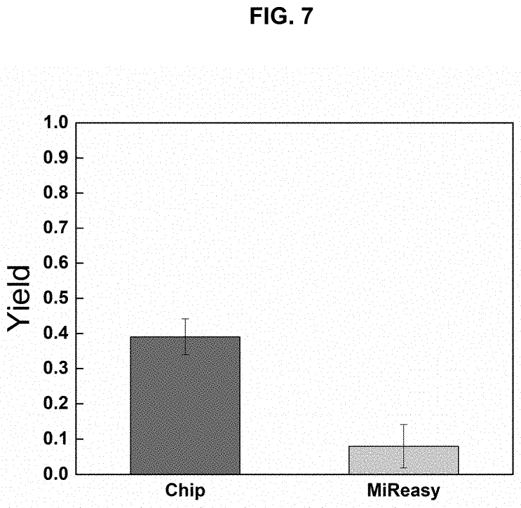

FIG. 8A. The change of cross channel current Ids over time at different flow rate with loaded buffer 0.1×PBS. V_{d}=1 V, V_{g}=80 V.

FIG. 8B. The change of cross channel current Ids over time at different flow rate with loaded buffer 0.4×PBS. V_{d}=1 V, V_{g}=80 V.

FIG. 9. Gel electrophoresis image of PCR amplicons of both treated and untreated E. coli DNA in 0.1×PBS with 20 mM calcium chloride.

FIG. 10A. Real-time fluorescence images show the isolation of dsDNA from a plasma sample at 4 min.

FIG. 10B. Real-time fluorescence images show the isolation of dsDNA from a plasma sample at 6 min.

FIG. 10C. Real-time fluorescence images show the isolation of dsDNA from a plasma sample at 8 min.

FIG. 10D. Real-time fluorescence images show the isolation of dsDNA from a plasma sample at 10 min.

FIG. 10E. Real-time fluorescence images show the isolation of dsDNA from a plasma sample at 12 min.

FIG. 10F. Real-time fluorescence images show the isolation of dsDNA from a plasma sample at 14 min.

### DETAILED DESCRIPTION

Described herein is a bifurcated continuous field-flow fractionation (BCFFF) chip for high-yield and high-throughput nucleic acid extraction and purification. BCFFF uses a membrane ionic transistor to sustain low-ionic strength in a localized region at a junction, such that the resulting high field can selectively isolate high-charge density nucleic acids from the main flow channel and insert them into a standardized buffer in a side channel that bifurcates from the junction. The BCFFF platform can be used for isolation of both long dsDNAs and short miRNAs, without changing the device configuration or the operation protocol. BCFFF results in high-efficiency (>85%) concentration-independent DNA extraction and 40% net qRT-PCR miRNA yield from plasma, which is significantly higher than any other commercial liquid and solid extraction technologies.

One embodiment described herein is an apparatus for isolating nucleic acids from a fluid sample comprising the nucleic acids, the apparatus comprising: an inlet, a first outlet, a second outlet and a microfluidic channel fluidly connecting the inlet, the first outlet and the second outlet, wherein the microfluidic channel includes a first channel portion extending from the inlet to a junction, a second channel portion extending from the junction to the first outlet, and a third channel portion extending from the junction to the second outlet; a cation exchange membrane (CEM) positioned across the second channel portion proximate to the junction; a plurality of electrodes adapted to generate an electric field at the junction. In one aspect, the plurality of electrodes include a first terminal positioned within the third channel portion, a second terminal positioned within the second channel portion and a third terminal positioned on the CEM. In another aspect, the microfluidic channel has a thickness between about 1 μm and about 100 μm.

Another embodiment described herein is a method for isolating nucleic acids from a fluid sample comprising the nucleic acids, the method comprising: providing the apparatus described herein; filling the microfluidic channel with a buffered solution comprising anions and cations; applying an electric field at the junction of about 0.1 to 10 V/mm thereby causing cations at the junction to migrate away from the junction through the CEM, and causing anions at the junction to migrate away from the junction towards the second outlet, and generating an ion depletion region at the junction; isolating nucleic acids from the fluid sample by: continuously loading the fluid sample into the microfluidic channel through the inlet and removing fluid from the first outlet such that fluids flow from the inlet to the first outlet at a flow rate that permits nucleic acids to electrophoretically migrate from the junction towards the second outlet due to the applied electric field, and to concentrate at the second outlet; and thereafter, collecting concentrated nucleic acids from the second outlet. In one aspect, the ion depletion region has an ionic strength less than about 1.0 mM. In another aspect, the dimension of the ion depletion region is inversely proportional to the applied electric field. In another aspect, the length of the ion depletion region is between about 0.1 mm and about 10 mm. In another aspect, the applied electric field and the flow rate are selected so as to permit at least 80% of the nucleic acids in the fluid sample to electrophoretically migrate from the junction towards the second outlet and concentrate at the second outlet. In another aspect, the flow rate is between about 0.1 μL/min and about 100 μL/min. In another aspect, the nucleic acids comprise one or more of ssDNA, dsDNA, rDNA, cDNA, ssRNA, dsRNA, rRNA, mRNA, tRNA, siRNA, or miRNA. In another aspect, the fluid sample further comprises one or more cationic molecules, and wherein the applied electric field and the flow rate cause the cationic molecules to electropheretically and hydrodynamically migrate from the junction towards the CEM. In another aspect, the cationic molecules comprise ions or proteins. In another aspect, the fluid sample further comprises anionic biomolecules other than nucleic acids, and wherein the flow rate causes the anionic biomolecules to hydrodynamically migrate from the junction towards the CEM.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any and all variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

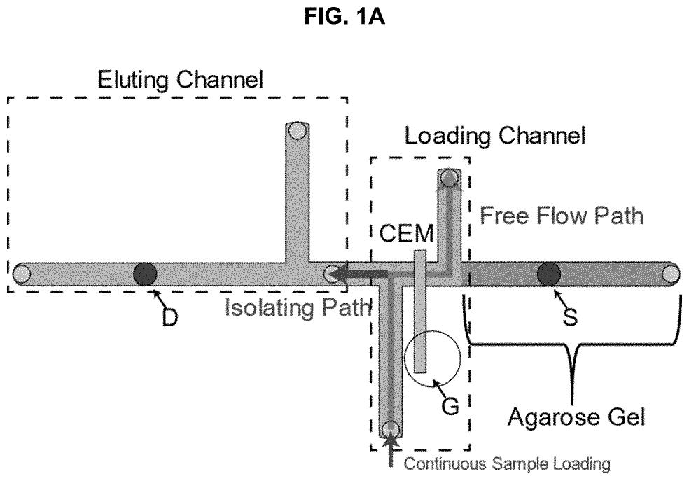

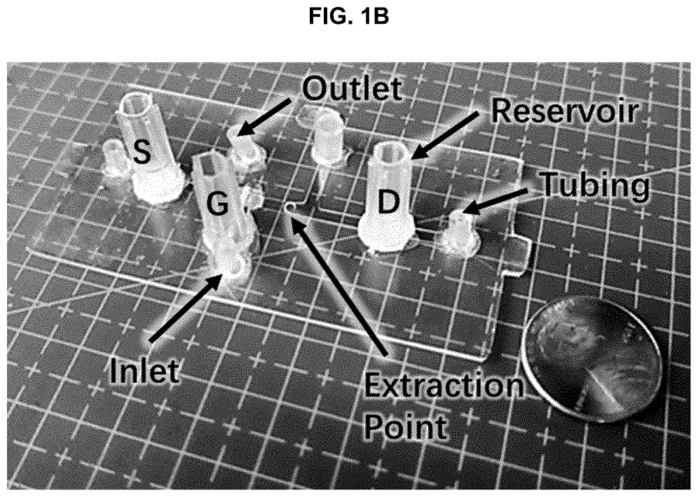

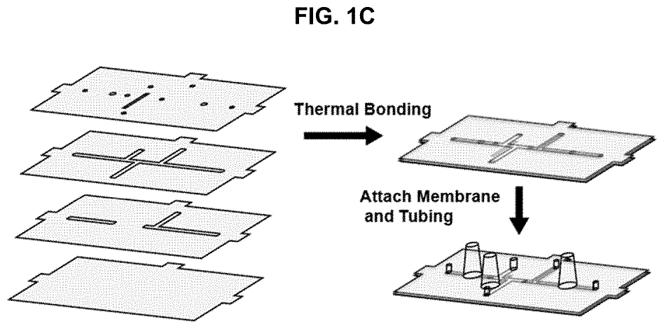

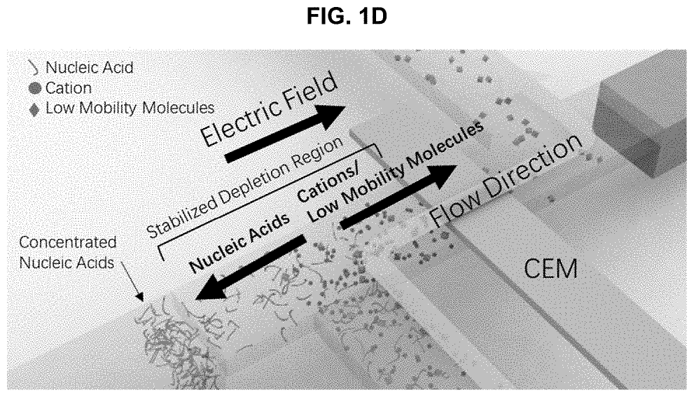

Fabrication of the Microfluidic Chips. The membrane transistor fabrication method is adopted from previous research of ionic transistor (Sun et al., Lab on a chip 2016, 16, 1171-7; Sun et al., Phys. Rev. Applied 2017, 7, 064024). As shown in FIG. 1C, the microfluidic BCFFF chip is fabricated by thermal bonding of four layers of polycarbonate (PC) sheets, whose low zeta potential minimizes electro-osmotic flow. For each layer, the patterns are cut on a plotter (Graphtec Cutting Pro FC7000MK2-60). The top 300 μm-thick layer consists of the openings of microchannels for fluidic connections and membrane attachment. The bifurcated channel pattern is on the second 100 μm-thick layer. Vortices are often observed at the ion-selective membrane and at the boundary of the ion-depleted region (the depletion layer) in our earlier work on gated ionic transistors (Sun et al., Lab on a chip 2016, 16, 1171-7; Sun et al., Phys. Rev. Applied 2017, 7, 064024). The thin second layer is to enhance viscous dissipation and to suppress these vortices. Earlier studies have shown that the vortices do not appear at this thickness (Yossifon and Chang, Physical Review Letters 2008, 101, 1-4; Yossifon and Chang, Physical Review Letters 2009, 103, 1-4). The third 300 μm-thick layer increases the volume of side channels to enhance the electric field under the cation-exchange membrane (CEM) by field focusing. The bottom 300 μm-thick layer is the substrate. These PC sheets are aligned and thermally bonded together at 173° C. for 30 min. A strip of CEM is sealed onto the chip with a UV curable glue (Acrifix 192). The extraction outlet is sealed with tape for easy extraction. The D and S openings on the chip are first covered with cut filter paper. Cut pipette tips as buffer reservoirs for electrical connection and Tygon tubing as fluidic inlets and outlets are fixed by the UV curable glue onto their designated places on the top of the chip. The device is filled with 0.1×PBS buffer. 1% agarose gel in 0.1×PBS is placed on the bottom of each reservoir and filled into the side channel under the S reservoir. The chip is left overnight to let the CEM swell properly before use.

Experiment Setup. During the experiment, all the openings of the chip are sealed except the inlet and outlet of the loading channel. Platinum wires are fixed in the G, D, S reservoirs on the microfluidic chip. External voltages are applied through these wires by Keithley 2636A Dual-Channel System SourceMeter Instrument controlled and monitored by custom MATLAB code. The samples are loaded on to the chip through the inlet of the loading channel by a Braintree syringe pump. After pretreatment, the outlet of the loading channel is sealed, 4 μL of purified sample is quickly collected from the extraction outlet with a pipette.

Fluorescent Visualization and Measurement. The real-time fluorescence images of pretreatment of ssDNA are taken by using an inverted epifluorescence microscope (Olympus 1X71) equipped with a mercury lamp and a high-speed camera (QImaging Retiga-EX). The visualization of on-chip pretreatment of dsDNA from plasma is performed on a customized dark-room platform equipped with a Dark Reader Transilluminator (Clare Chemical) to excite the fluorescent dye from the bottom of the microfluidic chip. The fluorescent signal is filtered and recorded at the top of the microfluidic chip by a Logitech C920 Webcam. SYBR® Green I Nucleic Acid Stain is purchased from Lonza (Cat #50513). Pierce™ Recombinant GFP Protein is purchased from Thermo Scientific™ (Cat #88899). The fluorescence measurement of pretreated samples is performed on Tecan Infinite M200 Plate Reader.

Plasma Samples. De-identified fresh human plasma samples were purchased from Zen-Bio Inc. The 10 mL samples were collected in tubes with EDTA anticoagulant. All samples were obtained following FDA-mandated testing for pathogens.

Quantification of Calcium Concentration. The concentration of calcium in each sample is measured by Inductively Coupled Plasma Optically Emitting Spectra (ICP-OES, Perkin Elmer Optima 8000).

E. coli DNA Amplification and Gel Electrophoresis. E. coli with pUC18 plasmid is purchased from Modern Biology Inc. The primers from IDT DNA are shown in Table 1. Pellet of E. coli is obtained by centrifugation at 5000×g for 10 min and resuspended in 1×PBS. The solution is put into 95° C. water bath for 10 min to thermally lyse the bacteria. PCR of E. coli DNA is carried out on a Bio-Rad MJ Mini. Each 20 μL reaction contained 2 μL of the sample, 10 μL SsoAdvanced Universal SYBR Green Supermix (Bio-Rad), 500 nM of forward primer, 500 nM of reverse primer, and 4 μL water. The following TaqMan thermocycling conditions were used: 10 min at 95° C., followed by 28 cycles of 95° C. for 60 s, 50° C. for 60 s, and 75° C. for 180 s. Electrophoresis of the amplicon is run at 80 V for 1 hour in 1.2% agarose gel. The gel was stained with SYBR® Green I Nucleic Acid Stain (Lonza) and visualized under a dark reader transilluminator (Clare Chemical).

DNA Quantification. As shown in Table 1, the oligos and DNA templates sequences are adapted from literature report (Haugland et al., Water research 2012, 46, 5989-6001) and purchased from IDT DNA. The dsDNA is obtained by purification of PCR products from the DNA template with QuickClean PCR Purification Kit (GenScript). To quantify the yield of DNA, triplicates of qPCR reactions were carried out on a StepOnePlus™ Real-Time PCR System (Applied Biosystems). The reaction contained 2 μL of the sample, 10 μL TaqMan™ Universal Master Mix II, no UNG (Qiagen), 500 nM of forward primer, 500 nM of reverse primer, 500 nM of TaqMan probe, and 2 μL RNase-free water in a final volume of 20 μL. The following TaqMan thermocycling conditions were used: 10 min at 95° C., followed by 45 cycles of 95° C. for 30 s and 60° C. for 60 s. The C_{q} values were acquired and analyzed using StepOne™ Software v2.3.

TABLE 1
Oligonucleotide and gene sequences used in this study

| Sequence Name | Sequence | SEQ IQ NO |
|---|---|---|
| Fluorescence-labeled | 5′-TAGCCCTAAAGCTATTTCGGAGAGAACCA-3′ | SEQ ID NO: 1 |
| ssDNA Sequence | | |
| Enterococcus | 5′-GAGAAATTCCAAACGAACTTG-3′ | SEQ ID NO: 2 |
| Forward Primer | | |
| Enterococcus | 5′-CAGTGCTCTACCTCCATCATT-3′ | SEQ ID NO: 3 |
| Reverse Primer | | |
| Enterococcus | [6-FAM]-5′-TGGTTCTCTCCGAAATAGCTTTAGGGCTATAMRA- | SEQ ID NO: 4 |
| TaqMan ® Probe | 3′ | |
| DNA Template for | 5′- | SEQ ID NO: 5 |
| dsDNA Fragment | TCATGCAAGTCGAGCGATGGAGAAATTCCAAACGAACTTGGGGGTT | |
| | CTGAGAGGAAGGTGGTAGAGCACTGTTTCGGCATCTGAGGAGCACG | |
| | AGACGGCAGGCTCGAGAATGATGGAGGTAGAGCACTGAAAAGGAAG | |
| | ATTAATACCGCATAGAGAATGTTATCACGGGAGACAAGTAGCGTGA | |
| | AGGATGACGG-3′* | |
| rDNA Forward Primer | 5′-ACGAATTCGTGCCAGCAGCCGCGGTAA-3′ | SEQ ID NO: 6 |
| rDNA Reverse Primer | 5′-TGGAATICGGTTACCTTGTTACGACTT-3′ | SEQ ID NO: 7 |
| Ampicillin-resistance | 5′-GCTCACCCAGAAACGCTGGTGAAAGTA-3′ | SEQ ID NO: 8 |
| gene Forward Primer | | |
| Ampicillin-resistance | 5′-CGCAACGTTGTTGCCATTGCTACAGGC-3′ | SEQ ID NO: 9 |
| gene Reverse Primer | | |

*Bold: forward primer sequence, underlined: complementary sequence of reverse primer.

MiRNA Quantification. For the quantification of miRNA, qRT-PCR was performed on each extracted sample. Reverse transcription was carried out using a miScript II RT Kit (Qiagen). A 20 μL reverse transcription reaction was prepared with 2 μL of eluted miRNA, 4 μL 5× miScript HiSpec Buffer (Qiagen), 2 μL 10× miScript Nucleics Mix (Qiagen), 10 μL RNase-free water, and 2 μL miScript Reverse Transcriptase Mix (Qiagen). The reaction was incubated at 37° C. for 60 min, followed by 95° C. for 5 min. The reverse transcription reaction was then diluted with 200 μL RNase-free water. Triplicates of qPCR reactions were carried out using the miScript SYBR Green PCR Kit (Qiagen). The reaction contained 2 μL diluted cDNA, 12.5 μL 2× QuantiTect® SYBR Green PCR Master Mix (Qiagen), 2.5 μL 10× miScript Universal Primer (Qiagen), 10× miScript Primer Assay (Qiagen) for the target miRNA, and 5.5 μL RNase-free water in a final volume of 25 μL. The reaction mixtures were incubated for 15 min at 95° C., followed by 45 cycles of 94° C. for 15 s, 55° C. for 30 s, and 70° C. for 30 s.

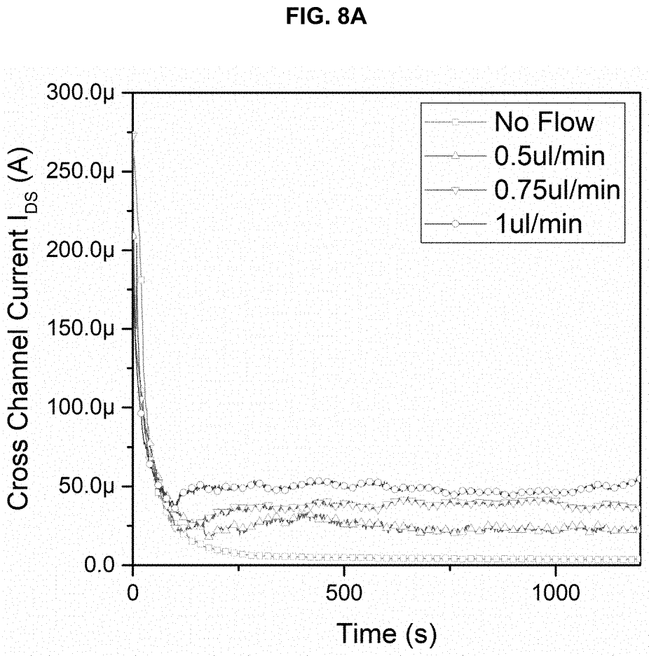

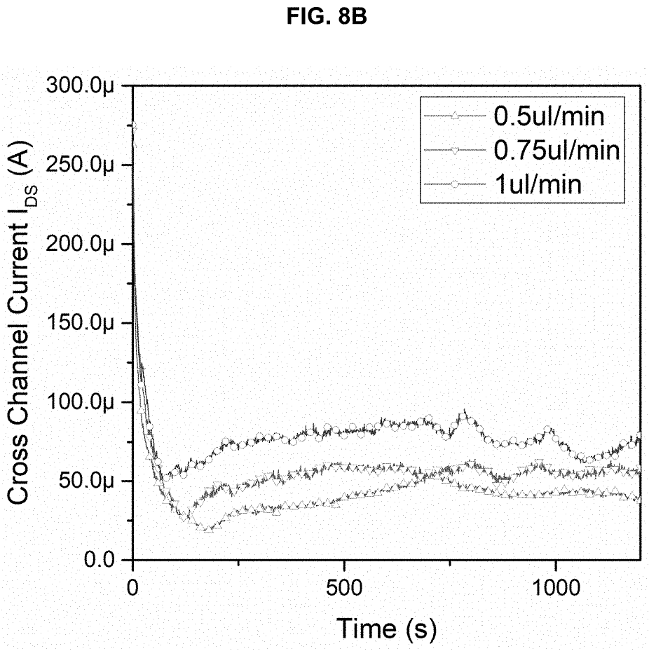

Stability of the Depletion Front. To investigate the influence of flow on the depletion generated by the CEM, ionic current going through the cross channel is recorded. Without flow, the current initially drops down following a diffusive √t law and then approaches a zero-current steady state. FIG. 8 shows that as the flow rate increases, a steady-state current is still observed, suggesting a stabilized depletion. However, a higher steady-state current under high flow rate indicates a lower resistance inside the depletion region and, consequently, a lower electric field, which is also true when the ionic strength of the loaded sample is increased. Low electric field cannot achieve high-yield extraction. There is hence an optimum flow rate for a given voltage.

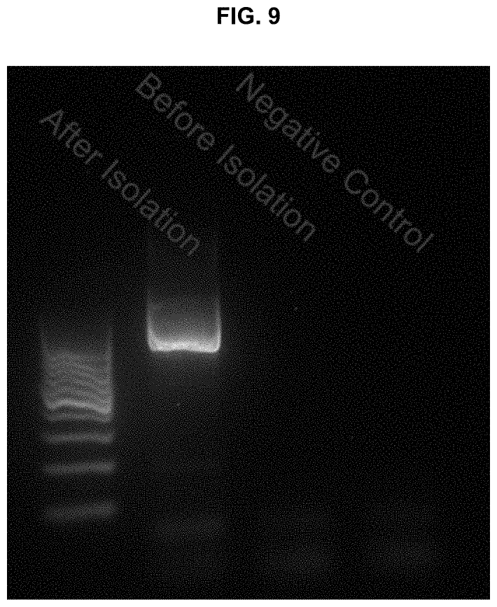

Proof-of-Concept of Calcium Removal by Purification of E. coli DNA from High Calcium Concentration Buffer. To prove the ability of extracting nucleic acid from inhibitors, a proof-of-concept experiment is done with thermally lysed E. coli spiked into 0.1×PBS buffer with 20 mM of added-in calcium chloride (Sigma-Aldrich). The E. coli (Modern Biology Inc. IND-21) is cultured in LB Broth Media (Teknova) for 2 days. The media is centrifuged at 5000×g for 15 minutes. The supernatant is removed, and the pellet is resuspended in 0.1×PBS buffer with 20 mM of added-in calcium chloride and thermally lysed in 95° C. water bath for 20 minutes. The lysed E. coli is pretreated with our device using the same DNA protocol described in the main article. PCR amplification of both pretreated and untreated sample is carried out using the primer sets and thermal cycling condition in the IND-21 kit targeting an ampicillin-resistance gene on plasmid pUC18. 1% agarose gel is prepared by mixing 0.5 g agarose I (Bioscience) and 1.2 μL SYBR® Green I Nucleic Acid Stain (Lonza) in 50 mL 1×TAE buffer (VWR). After casting the gel, 10 μL of amplicons of the sample is mixed with 2 μL of 10× loading buffer and loaded onto the gel. Electrophoresis was run at 120 V for 1 hour. The gel was visualized by Dark Reader Transilluminator (Clare Chemical). As shown in FIG. 9, successful amplification is achieved after isolation of DNA on BCFFF chip comparing to no target band for untreated sample.

### Example 2

### Principle of the On-Chip Nucleic Acid Extraction

The current adsorption-based solid-extraction technologies are plagued by low yield for short miRNAs (Jimenez et al., Anal Bioanal Chem 2018, 410, 1053-1060; Vigneron et al., Molecular Oncology 2016, 10, 981-992). For adsorption of polyelectrolyte, the critical substrate surface charge density for adsorption scales as 1/N (de Carvalho et al., Soft Matter 2015, 11, 4430-4443), where N is the length of the polyelectrolyte. Therefore, the yield of the column goes down at least proportionally with the length, with the all-important short miRNAs having the lowest yield. Different nucleic acid sequences also have different binding affinities to the substrate even if they have the same length (Kim et al., Mol. Cell 2012, 46, 893-895; Monleau et al., BMC Genomics 2014, 15, 395), which leads to variation in extraction efficiency and inaccuracy of the quantification. To the contrary, due to their high charge density, free-flow electrophoretic mobility of nucleic acids is higher than any other large biomolecule and beyond a critical length (>200 bp), a saturation mobility of 2×10⁻⁴ cm² V⁻¹ s⁻¹ to 4×10⁻⁴ cm² V⁻¹ s⁻¹ independent of concentration, size, and electric field strength is reached (Stellwagen et al., Biopolymers 1997, 42, 687-703; Grossman and Soane, Journal of Chromatography A 1991, 559, 257-266; Slater et al., Current Opinion in Biotechnology 2003, 14, 58-64). Interestingly, shorter miRNAs that are no more than a few persistence lengths long exhibit more hydrodynamic screening than electrostatic screening (Grass and Holm, J. Phys.: Condens. Matter 2008, 20, 494217), producing higher mobility than even longer nucleic acids and hence exhibit the highest electrophoretic mobility among all biomolecules. The high electrophoretic mobility of nucleic acid molecules, particularly short miRNAs, will be exploited in our BCFFF design, as a field-flow extraction design with an electric field is expected to have a better yield than solid-phase extraction or other field-flow designs with other fields, particularly for short miRNAs.

Even though miRNAs should have the largest free-space electrophoretic mobility of all biomolecules, their mobility should still be significantly lower than that of ions, particularly the ubiquitous divalent cations like Ca²⁺ that are also PCR inhibitors. Hence, the field-flow fractionation design must also reject counter-ions to nucleic acids, particularly multivalent cations. This issue motivates the bifurcated-channel design in BCFFF, as shown in FIG. 1D, such that the nucleic acids and their counterions can be separated because of their opposite electrophoretic directions.

Even with the high mobility of 4×10⁻⁴ cm² V⁻¹ s⁻¹ for miRNA, a high electric field (˜100 V/cm) is still required for their extraction into the bifurcated channel, during passage through the typical millimeter-length of the junction, at the standard high-throughput linear velocity of mm/s. Such a high field in a typical physiological fluid with ion strength of ≥100 mM would produce a large ionic current and a high Ohmic heating rate of tens of mW/mm², causing bubble formation and pH changes due to electrochemical reactions at elevated temperatures and voltages. Our solution is to locally deplete the ionic strength to below mM, which is the principle behind many recent electrokinetic chip designs which use ion-selective membrane for on-chip ion concentration depletion (Quist et al., Anal. Chem. 2011, 83, 7910-7915; Quist et al., Anal. Chem. 2012, 84, 9065-9071; Marczak et al., Biosensors and Bioelectronics 2016, 86, 840-848). The electric field in the ion-depleted region is inversely proportional to the dimension of the depleted region. Hence, ideally, the depletion region should be localized just at the junction of the bifurcated-main channel to sustain a high field at the working position. Any fluctuation in the length of the depletion zone would corrupt and render inconsistent the yield of the extraction pretreatment process. However, the extent and intensity of depletion are difficult to control and an excessively large depletion zone can reduce the field intensity at the desired location. Our recent work uses a 3-terminal ionic transistor design to stabilize the depletion front at a designated location (Sun and Chang, Lab on a chip 2016, 16, 1171-7; Sun et al., Phys. Rev. Applied 2017, 7, 064024). This design is ideal for continuous isolation of target from the sample flow to standard buffer in the cross channel, where the high field of the ion depletion region is only needed at the junction between of the main channel with the eluting bifurcated channel. As shown in FIG. 1C, an ionic transistor with a cation exchange membrane (CEM) is implemented in order to drive counter-ions such that negatively-charged nucleic acids move away from the membrane, against the flow, towards the eluting channel. The source terminal “S” is fixed to 0 V. And, the size of the depletion region can be adjusted by tuning the ratio between the draining potential (V_{d}) at “0” terminal and gating potential (V_{g}) at the “G” terminal. The sample is introduced into the depletion region generated by the ionic transistor through a perpendicularly intersected loading channel continuously with a syringe pump. External voltages are designed to extend the depletion zone from the loading channel to the right edge of the eluting channel (FIG. 1C). Inside the depletion region, high-mobility anionic molecules like nucleic acids are driven by the electric field towards to the elution channel, while low-mobility and cationic molecules are driven away from the elution channel, by the combination of electric force and hydrodynamic drag.

### Example 3

### Estimation of Nucleic Acid Extraction by Fluorescence

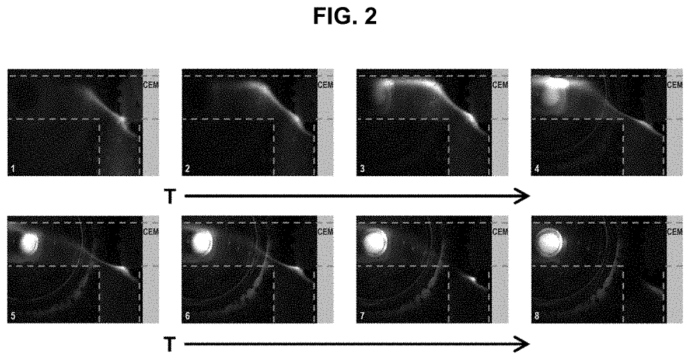

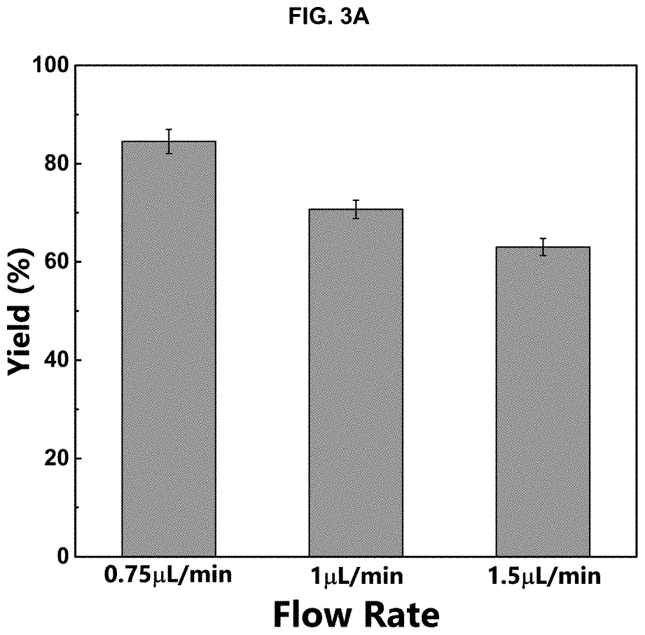

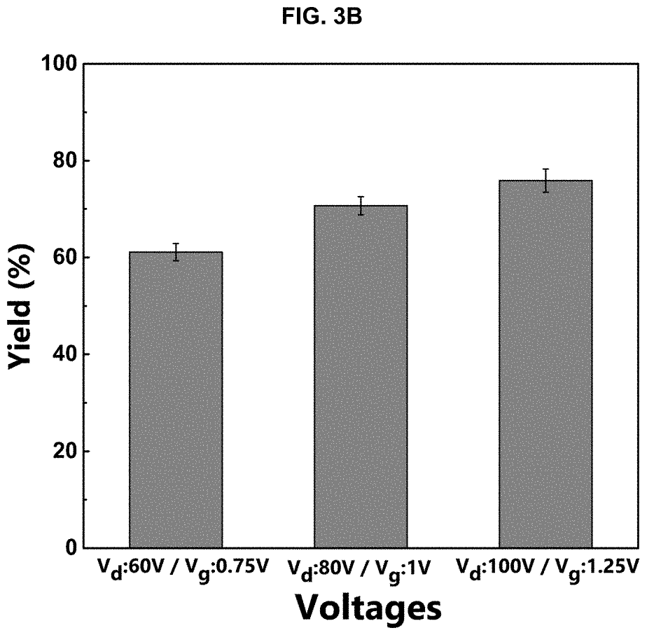

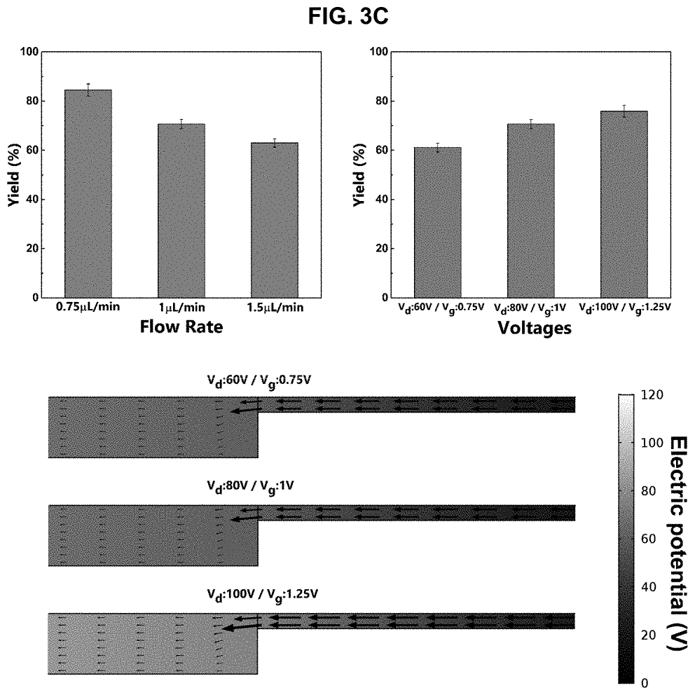

The extraction efficiency of this method is first quantified by comparing the total fluorescence of fluorescence-labelled ssDNA before and after the on-chip isolation. Such efficiency is dictated by the electric field and the hydrodynamic drag locally at the junction, which are independently controlled by external voltages and the flow rate applied to the systems. Inside the depletion region which is stabilized at the junction by fixing V_{d}/V_{g} at 80 (FIG. 1C) (Sun et al., Phys. Rev. Applied 2017, 7, 064024), the sample loading flow is in the opposite direction of the electric field. Hence, there is a competition between electrophoretic velocity and convective velocity of the target. The electrophoretic velocity is defined by U_{electrophoresis}=μₑE, where μₑ is the electrophoretic mobility and E is the electric field. The convective velocity, on the other hand, is specified by the flow rate applied. The applied electric field needs to be high enough to push the target nucleic acids towards the eluting channel. As demonstrated in FIG. 2, the fluorescence-labelled ssDNA is driven out of the flow by the electric field and stabilized at the designated extraction point. FIG. 3 shows the experimental data exactly as expected—the extraction efficiency increases with higher voltage and decreases with a higher flow rate. In all cases, this method shows a promising isolation yield—greater than 50% in the tested combination of parameters and can reach as high as 85% once optimized for a particular flow rate, sample ionic strength, and channel geometry. The effect of increasing ionic strength of the loaded sample should be similar to that of decreasing voltages since smaller resistance of the depletion region can lower down the potential drop inside. Thus, there is a trade-off between the throughput (flow rate and ionic strength) and the extraction efficiency of the system. Nucleic acids are concentrated locally at the designated location. With the continuous flow design, the enrichment of target molecules can be done for an arbitrary volume of the sample—the trade-off is the pretreatment time, as FIG. 3 indicates that the flow rate should not exceed a certain critical value of roughly 0.75 μL/min. The extraction yield is also independent of DNA copy number. Unlike, batch liquid and solid extraction, saturation that corrupts high copy number samples is not an issue. A washing step that is often responsible for low extraction yield of low copy number samples is also absent. We will subsequently establish through serial dilution that our extraction yield is indeed concentration independent.

### Example 4

### Removal of Major PCR Inhibitors

One of the major inhibitors of PCR amplification is calcium ion, particularly in urine and plasma samples. Calcium ions can compete with magnesium ions, which is a cofactor of the polymerase reaction (Schrader et al., J Appl Microbiol 2012, 113, 1014-1026), during the latter's binding with the DNA polymerase. To evaluate the calcium removal efficiency of our device, we perform pretreatment experiments on 1 μM ssDNA spiked in 10 μL 0.5×PBS samples with −2 mM of calcium ions. These concentration levels are comparable to that in plasma and can cause inaccurate quantification or total inhibition of PCR amplification (Schrader et al., J Appl Microbiol 2012, 113, 1014-1026). After pretreated with the optimized protocol (V_{d}=1 V, V_{g}=80 V, flow rate=0.75 μL/min). FIG. 3A shows that the calcium concentration in the eluted sample drops by three orders of magnitude, regardless of its initial concentration. At this low calcium concentration, PCR can be directly performed with the eluted DNA sample. A more extreme proof-of-concept experiment is done with E. coli DNA from lysed bacteria with 20 mM spiked-in calcium chloride. As shown in FIG. 9, where the PCR products of both treated and untreated sample are run on the gel, a positive result is only achieved with the treated sample while no amplification is observed with the untreated sample.

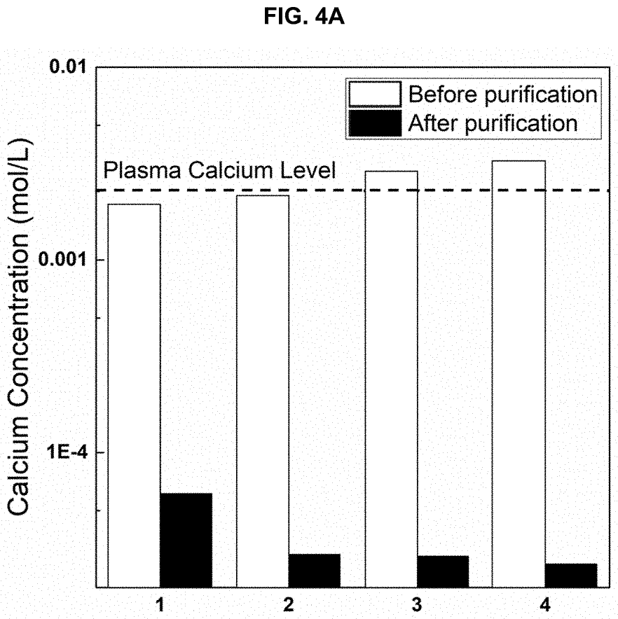

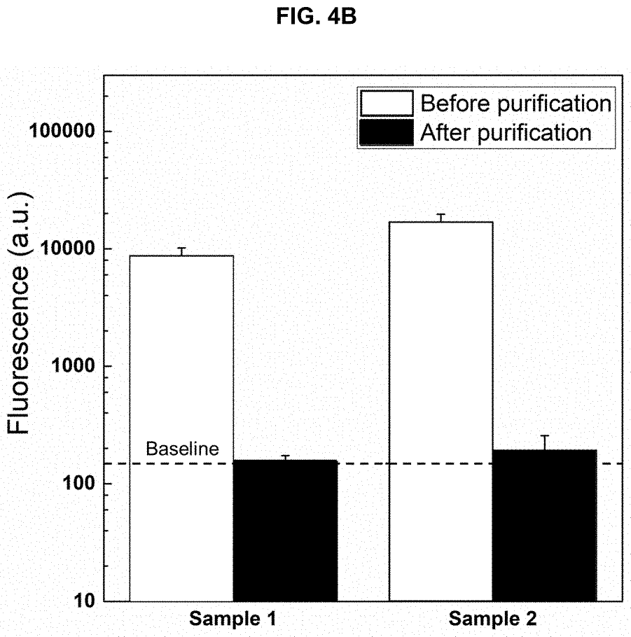

Another category of inhibitor is protein. Unlike nucleic acids, which are strongly negatively charged, most proteins are weakly charged, and their polarity can be either positive or negative depending on their isoelectric point. The positively charged proteins can be easily removed by our system just as other cationic molecules. For negatively charged proteins, their mobilities are usually much smaller than that of nucleic acids (Stellwagen et al., Biopolymers 1997, 42, 687-703; Chun and Lee, Colloids and Surfaces A: Physicochemical and Engineering Aspects 2008, 318, 191-198); thus, they tend to be dragged away by the flow instead of being collected into the eluting channel by the electric field. Here we use GFP molecules to validate the removal of protein using our device. The removal efficiency is demonstrated by loading 0.5×PBS spiked with GFP onto the pretreatment chip and running optimized protocol (V_{d}=1 V, V_{g}=80 V, Flow rate=0.75 μL/min) for nucleic acid extraction. The recombinant GFP is negatively charged in the physiological environment and the depletion region. However, because of its low electrophoretic mobility, it can be successfully separated from the isolated nucleic acids under the hydrodynamic drag force. As shown in FIG. 4B, the measured fluorescence signals from the eluted sample are very close to the baseline, which indicates almost none of the GFP molecules are collected into the eluting channel under this condition.

### Example 5

Amplification of DNA Extracted from Spiked Plasma Sample

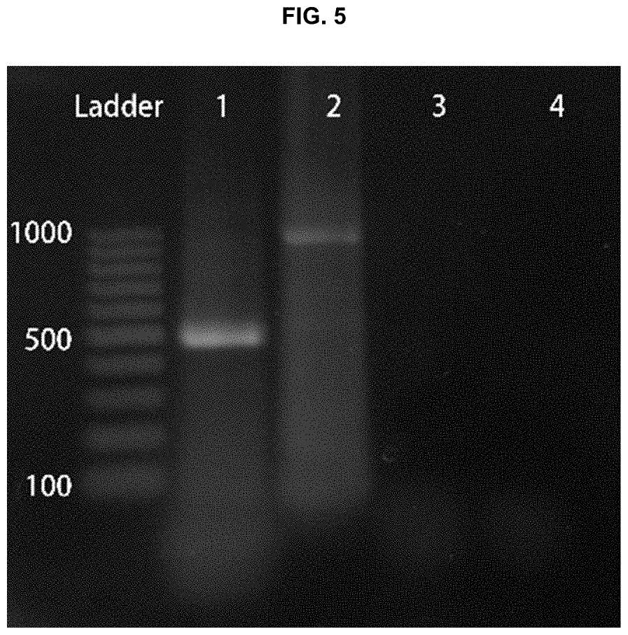

The ability of our device to extract and purify long DNA is verified with E. coli pUC18 plasmid spiked into human plasma samples. 10 μL of lysed E. coli in 1×PBS is spiked into 190 μL of human plasma. Human plasma contains 60-80 mg/mL of total protein and is the most heterogeneous sample for molecular detection and quantification (Walker et al., Clinical Methods: The History, Physical, and Laboratory Examinations, Butterworths, Boston, 3rd edn., 1990). After applying the voltage (V_{d}=1 V, V_{g}=80 V) and stabilizing the current, 30 μL of the prepared sample is loaded onto to the device at a flow rate of 0.75 μL/min followed by 5 μL of 0.1×PBS. 4 μL of fluid is eluted from the chip for PCR amplification. FIG. 5 shows the PCR result on the gel. Two targets—rDNA (550 bp product) and Ampicillin-resistance gene (1020 bp product) are amplified separately. The successful amplification of these two long targets, compared to the absence of target bands for untreated samples, indicates that our device is capable of purifying long DNA from plasma for PCR. Moreover, secondary amplicon is absent in the gel image, which suggests long DNA molecules are intact during the pre-treatment.

### Example 6

Quantification of Extracted DNA from Plasma

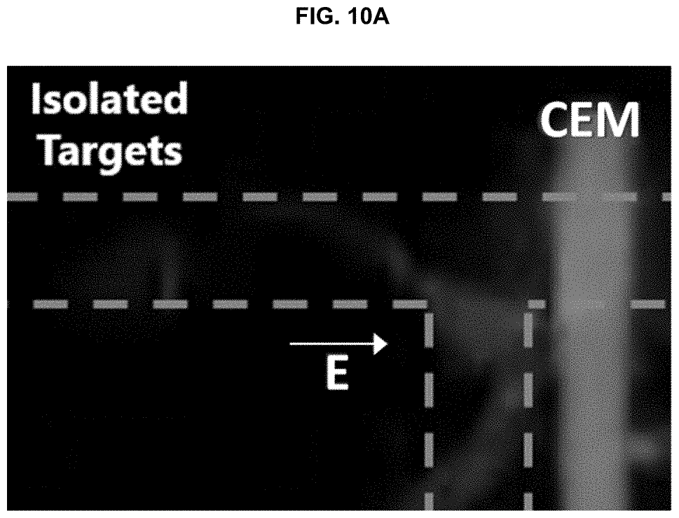

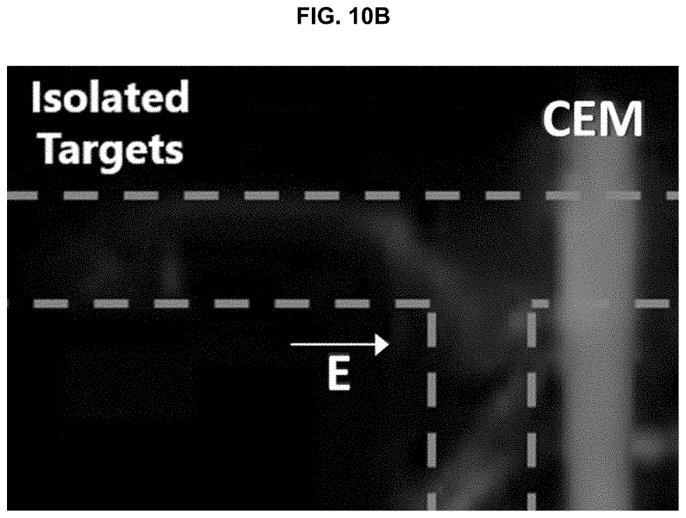

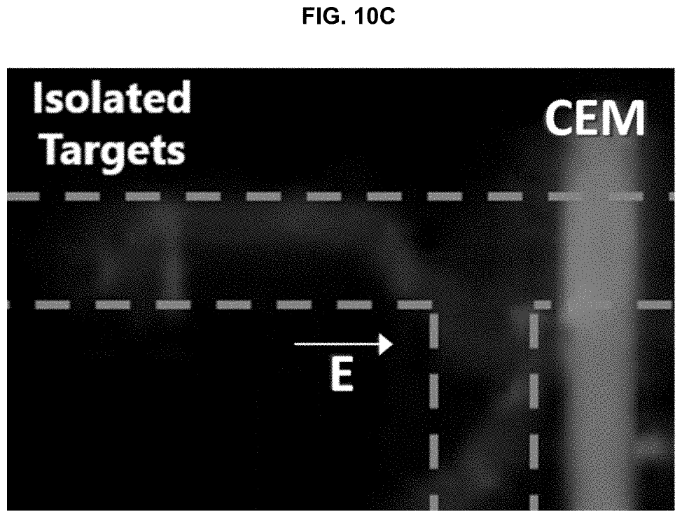

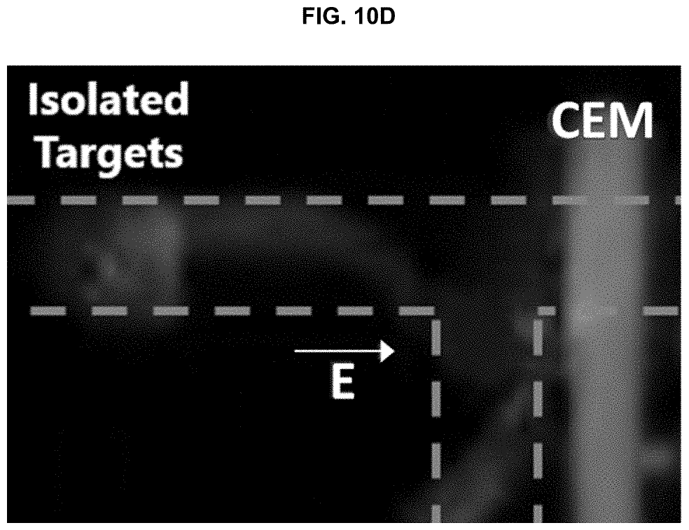

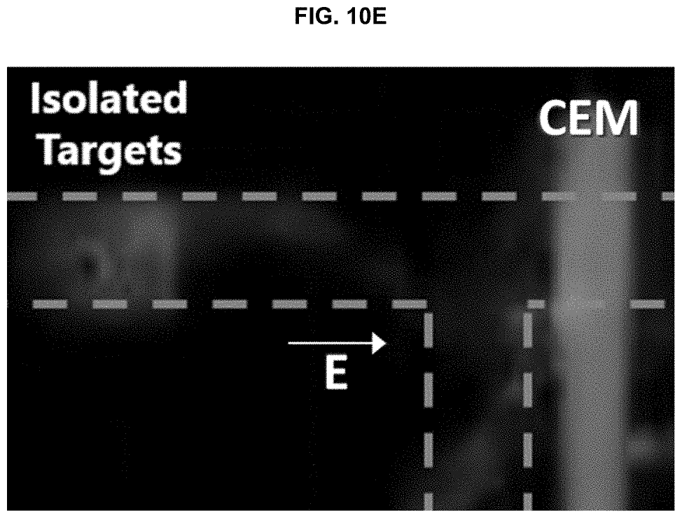

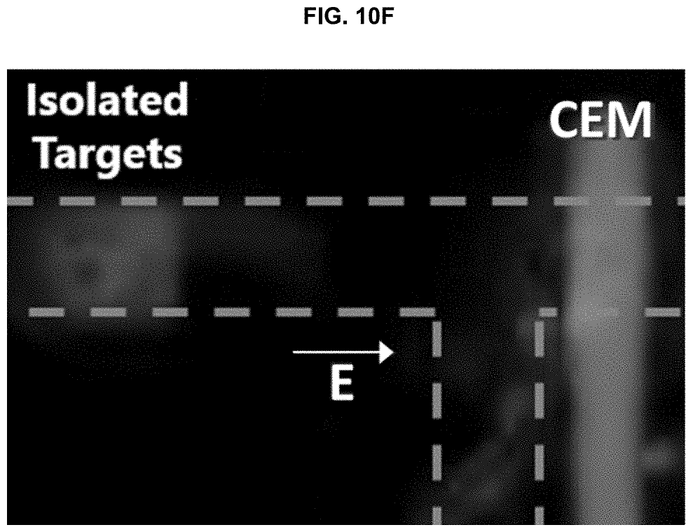

To quantify the yield of extracted nucleic acids and inhibitor removal efficiency of our BCFFF device, we perform pre-treatment of human plasma spiked with enterococcus DNA fragments. The 111-bp enterococcus DNA is exogenous for human and suitable as a spiked-in control to evaluate purification efficiency. To achieve a higher yield with acceptable throughput, the spiked plasma is diluted by DI water of the same volume. 1 μM of dsDNA sample is labelled with SYBR dye to visualize the process (FIG. 10). 10 μL of the sample, spiked with 1×10⁶ copies/μL of dsDNA, is pretreated by the chip with a loading flow rate of 0.75 μL/min followed by 5 μL of 0.1×PBS to flush the remaining plug of the sample inside the loading channel. The entire pre-treatment process hence takes about 20 minutes. The successful isolation of nucleic acids is confirmed by fluorescence imaging of the BCFFF chip.

We evaluated the purification from inhibitors of the eluted sample by examining the PCR efficiency of the spiked enterococcus DNA. In PCR experiments, impurity in the sample could lead to low PCR efficiency and nonlinearity during serial dilution of the sample. A series of dilution is carried out on the eluted sample. The qPCR result of these diluted samples is compared with both the result from a series of dilution of inhibitor-free control in DI water (FIG. 5A). The amplification efficiency of the PCR reaction is evaluated by the slope of the fitting curve. An average ΔC_{q} of 4 is found for target concentrations off by a factor of 10. The amplification efficiency is hence estimated to be larger than 60% (˜62%). In contrast, no amplification is observed for the untreated plasma sample after 45 cycles, suggesting total inhibition in the inhibitor-rich plasma. After pretreatment, however, the slope of the fitting curve from the isolated nucleic acids is close to that of the inhibitor-free control, which demonstrates successful removal of inhibitory molecules from plasma with our pretreatment unit. With the estimated 85% extraction yield, we estimate the inhibitor-free PCR reaction yield is 80% and can be improved with a better selection of primers and thermal protocol.

A series of spiked plasma sample is pretreated on the chip following the same protocol described above (FIG. 5B). The result shows good linearity between C_{q} value and logarithm of the copy number, suggesting constant extraction efficiency over a three-decade range of the target concentration. Moreover, a similar ΔC_{q} of 4 is found for a 10-fold change in target copy number as in the serially diluted samples in FIG. 5A—for all three decades of concentrations.

This suggests we have removed all the inhibitors in both the undiluted and diluted plasma samples. The BCFFF pre-treatment module hence can effectively remove all the plasma inhibitors, independent of the target copy number. As far as we know, it is the first pre-treatment unit whose extraction yield is concentration-independent.

### Example 7

On-Chip miRNA Extraction

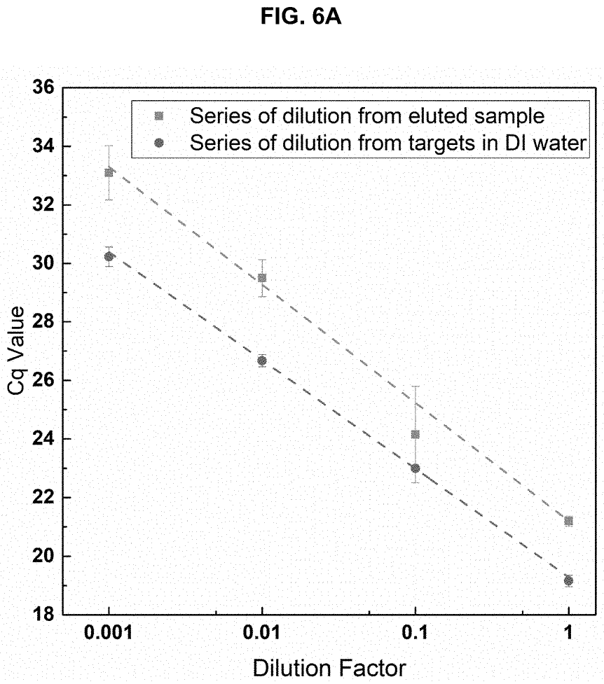

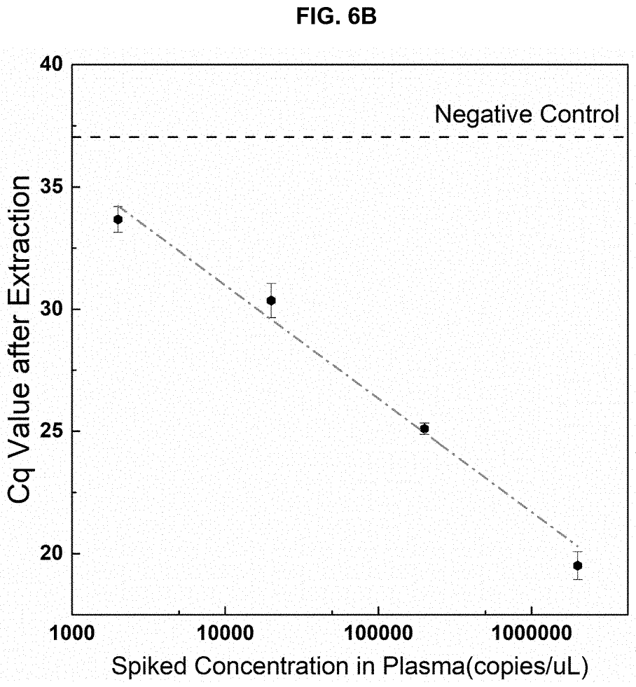

Not only can our BCFFF device purify large DNA fragments, but it also shows excellent performance for isolation of small miRNAs. The extraction efficiency of miRNA by our chip is compared with the commercial kit. Samples are prepared by spiking 3.5 μL of 1.6×10⁸ copies/μL of the synthetic 22-base cel-mir-39 into 200 μL plasma. MiRNA is isolated from 20 μL sample with the same protocol optimized for DNA extraction. For comparison, Commercial kit (Qiagen miReasy Serum/Plasma Kit) is used on 100 μL samples with the protocol described in the manual. Reverse transcription is carried out on the eluted sample, and qPCR analysis is used to quantify the number of miRNAs and to calculate extraction efficiency. As shown in FIG. 6, overall reverse-transcription yield of over 40% is obtained, compared to less than 10% from the commercial kit, with an estimated 50% yield for the reverse transcription step and a near-100% yield for the PCR reaction for both. It is lower than the 60% to 80% yield of the buffer, which can be corrected by applying a higher electric field.

1. An apparatus for isolating nucleic acids from a fluid sample comprising the nucleic acids, the apparatus comprising:
an inlet, a first outlet, a second outlet and a microfluidic channel fluidly connecting the inlet, the first outlet and the second outlet, wherein the microfluidic channel includes a first channel portion extending from the inlet to a junction, a second channel portion extending from the junction to the first outlet, and a third channel portion extending from the junction to the second outlet;
a cation exchange membrane (CEM) positioned across the second channel portion proximate to the junction;
a plurality of electrodes adapted to generate an electric field at the junction.

2. The apparatus of claim 1, wherein the plurality of electrodes include a first terminal positioned within the third channel portion, a second terminal positioned within the second channel portion and a third terminal positioned on the CEM.

3. The apparatus of claim 1, wherein the microfluidic channel has a thickness between about 1 μm and about 100 μm.

4. A method for isolating nucleic acids from a fluid sample comprising the nucleic acids, the method comprising:
providing the apparatus of claim 1;
filling the microfluidic channel with a buffered solution comprising anions and cations;
applying an electric field at the junction of about 0.1 to 10 V/mm thereby causing cations at the junction to migrate away from the junction through the CEM, and causing anions at the junction to migrate away from the junction towards the second outlet, and generating an ion depletion region at the junction;
isolating nucleic acids from the fluid sample by:
continuously loading the fluid sample into the microfluidic channel through the inlet and removing fluid from the first outlet such that fluids flow from the inlet to the first outlet at a flow rate that permits nucleic acids to electrophoretically migrate from the junction towards the second outlet due to the applied electric field, and to concentrate at the second outlet; and
thereafter, collecting concentrated nucleic acids from the second outlet.

5. The method of claim 4, wherein the ion depletion region has an ionic strength less than about 1.0 mM.

6. The method of claim 4, wherein the dimension of the ion depletion region is inversely proportional to the applied electric field.

7. The method of claim 4, wherein the length of the ion depletion region is between about 0.1 mm and about 10 mm.

8. The method of claim 4, wherein the applied electric field and the flow rate are selected so as to permit at least 80% of the nucleic acids in the fluid sample to electrophoretically migrate from the junction towards the second outlet and concentrate at the second outlet.

9. The method of claim 4, wherein the flow rate is between about 0.1 μL/min and about 100 μL/min.

10. The method of claim 4, wherein the nucleic acids comprise one or more of ssDNA, dsDNA, rDNA, cDNA, ssRNA, dsRNA, rRNA, mRNA, tRNA, siRNA, or miRNA.

11. The method of claim 4, wherein the fluid sample further comprises one or more cationic molecules, and wherein the applied electric field and the flow rate cause the cationic molecules to electropheretically and hydrodynamically migrate from the junction towards the CEM.

12. The method of claim 11, wherein the cationic molecules comprise ions or proteins.

13. The method of claim 4, wherein the fluid sample further comprises anionic biomolecules other than nucleic acids, and wherein the flow rate causes the anionic biomolecules to hydrodynamically migrate from the junction towards the CEM.
